(19) United States
(12) Patent Application Publication
(10) Pub. No.: US 2022/0228225 A1
(43) Pub. Date: Jul. 21, 2022

(54) SNP MARKER COMBINATION AND IDENTIFICATION METHOD FOR PUDONG WHITE PIGS AND RAW MEAT PRODUCTS

(71) Applicant: ZHEJIANG UNIVERSITY, Hangzhou City, Zhejiang Province (CN)

(72) Inventors: Yuchun PAN, Hangzhou City, Zhejiang Province (CN); Qishan WANG, Hangzhou City, Zhejiang Province (CN); Zhe ZHANG, Hangzhou City, Zhejiang Province (CN); Zhen WANG, Hangzhou City, Zhejiang Province (CN)

(21) Appl. No.: 17/042,122

(22) PCT Filed: Dec. 31, 2019
(86) PCT No.: PCT/CN2019/130973

§ 371 Date: Sep. 26, 2020

(30) Foreign Application Priority Data
May 30, 2019 (CN) 201910463234.2

(51) Int. Cl. C12Q 1/6888

(52) U.S. Cl. CPC C12Q 1/6888; C12Q 2600/156

(57) ABSTRACT

The present invention discloses a SNP marker combination and an identification method for Pudong white pigs and raw meat products thereof, including: extracting genomic DNAs of raw pork or meat products, performing agarose gel electrophoresis and Sanger sequencing after PCR amplification, and identifying Pudong white pigs and meat products thereof according to SNP genotypes of characteristic loci of sequencing results; as for the Sanger sequencing, its identification loci are as follows: specific mutations occur at loci of pig18-52722267, pig8-146130825, pig9-10041850 and pig13-213464983. The present invention solves the problem that there is no identification method related to Pudong white pigs and meat products thereof in the prior art.

### TECHNICAL FIELD

The present invention relates to the field of food safety monitoring, and in particular to a SNP marker combination and an identification method for Pudong white pigs and raw meat products.

### BACKGROUND

Pudong white pigs are mainly distributed in Nanhui and Chuansha of Shanghai. They are characterized by a high reproduction rate and early sexual maturity, with an average of about 12 litters per birth. After castration, they are quiet and motionless, and suitable for soft-ring breeding. Before 1950s, they were the dominant breed in Chuansha County. Because of their delicious meat, they are widely welcomed by consumers. Pudong white pigs are similar in shape and appearance to Western Landrace and Yorkshire pigs, and there are cases in which Western pig breeds pretend to be Pudong white pigs in production. On the other hand, the distribution area of Pudong white pigs overlaps with that of Taihu Pigs (Erhualian Pigs, Meishan Pigs, Fengjing Pigs Shawutou Pigs, Mi Pigs and Jiaxing Black Pigs), which leads to confusion in production. It is very difficult to distinguish Pudong white pigs from other breeds of pigs by traditional methods, especially the slaughtered divided pigs and their processed meat products. With the development of sequencing technology, molecular markers have developed from the first generation of restriction fragment length polymorphism (RFLP) and the second generation of variable number Simple Sequence Repeat (SSR) to single nucleotide polymorphism (SNP). Compared with the previous two generations, the third-generation molecular marker SNP has the advantages of abundant variation, low requirement for DNA samples, high stability, accurate determination, simple detection method and high throughput. At present, the third-generation molecular marker SNP has been widely used in paternity testing, identification of animal and plant varieties (strains), genetic breeding and other fields.

### SUMMARY

The purpose of the present invention is to provide a SNP marker combination and an identification method for identifying Pudong white pigs and raw meat products thereof, aiming at the defects of the prior art. The third generation molecular marker identification and Sanger sequencing technology are used to identify Pudong white pigs and raw meat products thereof, which solves the problem that there is no identification method related to Pudong white pigs and their meat products in the prior art, and provides a method and related special primers for identifying Pudong white pigs and meat products thereof with accurate results, simple operations and a low price.

The purpose of the present invention is realized by the following technical solution:

A SNP marker combination for Pudong white pigs and raw meat products comprises pig18-52722267, pig8-146130825, pig9-10041850 and pig13-213464983, wherein the pig18-52722267 represents a 52722266^{th} locus of pig chromosome No. 18, the pig8-146130825 represents a 146130825^{th} locus of pig chromosome No. 8, the pig9-10041850 represents a 10041850^{th} locus of pig chromosome No. 9, and the pig13-213464983 represents a 213464983^{th} locus of pig chromosome No. 13.

Furthermore, a method for identifying Pudong white pigs and raw meat products thereof based on the SNP marker combination comprises : firstly extracting genomic DNAs of a raw pork or meat product to be identified, then performing agarose gel electrophoresis and Sanger sequencing after PCR amplification to obtain information of each detection locus of the SNP marker combination, wherein a Pudong white pig and a meat product thereof are identified when specific mutations occur at any three loci of the SNP marker combination.

Furthermore, as for the PCR amplification, sequences of upstream and downstream primers of the pig18-52722267 are shown in SEQ ID No.1 and SEQ ID No.2, sequences of upstream and downstream primers of the pig8-146130825 are shown in SEQ ID No.3 and SEQ ID No.4, sequences of upstream and downstream primers of the pig9-10041850 are shown in SEQ ID No.5 and SEQ ID No.6, and sequences of upstream and downstream primers of the pig13-2134664983 are shown in SEQ ID No. 7 and SEQ ID No.8.

Furthermore, comparison information of identification loci of sequencing products is shown in the following table:

| SNP | REF | ALT |
|---|---|---|
| pig18-52722267 | T | C |
| pig8-146130825 | G | T |
| pig9-10041850 | G | A |
| pig13-213464983 | C | T |

in which, REF represents a reference genotype and ALT represents a mutation genotype.

Furthermore, the information of identification loci is: mutation genotypes and reference genotypes of detection loci; when a reference genotype appears in a detection locus of a pig to be detected, the locus is determined to have no identification significance; and when a mutation genotype appears in a detection locus of the pig to be detected, the locus is determined to have identification significance.

The beneficial effect of the present invention is: compared with the prior art, the present invention takes the unique SNP loci of Pudong white pigs as the identification basis, studies the identification method of Pudong white pigs from the molecular level, and takes Sanger sequencing as the main molecular identification method, which can distinguish Pudong white pigs from common western pig breeds and local pig breeds in Taihu Basin, for example, small Meishan pigs, Fengjing pigs and Middle Meishan pigs, Erhualian pigs, Jiaxing black pigs, Mi pigs, Shawutou pigs, Landraces, Yorkshire pigs, Durocs, Pietrains, Barkshire, etc.

### DESCRIPTION OF EMBODIMENTS

The specific embodiments of the present invention will be described in further detail below.

The present invention relates to a SNP marker combination of Pudong white pigs and raw meat products, comprising pig18-52722267, pig8-146130825, pig9-10041850 and pig13-213464983, wherein the pig18-52722267 represents the 52722266^{th} locus of pig chromosome No. 18, the pig8-146130825 represents the 146130825^{th} locus of pig chromosome No. 8, the pig9-10041850 represents the 10041850^{th} locus of pig chromosome No. 9, and the pig13-213464983 represents the 213464983^{th} locus of pig chromosome No. 13.

The present invention provides a method for identifying Pudong white pigs and raw meat products thereof based on the above SNP marker combination, comprising the following steps of: firstly extracting genomic DNA of raw pork or meat products to be identified, then performing agarose gel electrophoresis and Sanger sequencing after PCR amplification to obtain information of each detection locus of the SNP marker combination, wherein a Pudong white pig and a meat product thereof are identified when specific mutations occur at any three loci of the SNP marker combination.

The primers involved in the PCR amplification are shown in Table 1:

TABLE 1
Information about Primers and Products of Amplification Loci

| SNP | F | R | length | F'-position |
|---|---|---|---|---|
| pig18-52722267 | SEQ ID NO. 1: | SEQ ID NO. 2: | 314 | 160 |
| | GCGTTTTGGGGACTCGTGATA | ACTCTGCCGTTTCTCCTCCTA | | |
| pig8-146130825 | SEQ ID NO. 3: | SEQ ID NO. 4: | | |
| | AGAGGAGCGGGGTCTTTGC | CGTTGTCAACTTTTGTCTACCT | 525 | 119 |
| | | CA | | |
| pig9-10041850 | SEQ ID NO. 5: | SEQ ID NO. 6: | 627 | 284 |
| | TAGATGTGAGCCCCAGCAGTT | ACTTCTGCTCCCCGCACCG | | |
| pig13-213464983 | SEQ ID NO. 7: | SEQ ID NO. 8: | 177 | 71 |
| | CACTCAGGATATTCACAATCTGG | TTAAAACGACCACGGCAACTC | | |

In the table, F represents an upstream primer, R represents a downstream primer, length represents the length of a standard product, and F′-position represents the position of a SNP locus in an amplification product.

In the PCR amplification, the reaction system is template DNA of 1 ng/uL, a primer of 1 uL, H₂O of 3.8 uL and 2× Taq PCR masrer mix of 50 ul; and/or the reaction procedure of the PCR reaction is: pre-denaturation at 95C° for 2 minutes, pre-denaturation at 95C.° for 30 seconds, annealing at 60C.° for 30 seconds, extension at 72C.° for 1 minute, with a cycle number of 30 times, and extension at 72C.° for another 10 min minutes.

The mass concentration of the agarose gel is 2%.

The strip length of the gel electrophoresis is required as shown in Table 1.

The comparison information of identification loci of sequencing products is shown in Table 2:

TABLE 2
Comparison Information of Identification
Loci of Sequencing Products

| SNP | REF | ALT |
|---|---|---|
| pig18-52722267 | T | C |
| pig8-146130825 | G | T |
| pig9-10041850 | G | A |
| pig13-213464983 | C | T |

in which, REF represents a reference genotype and ALT represents a mutation genotype.

Table 2 shows the mutation genotype and reference genotype of each detection locus. A detection locus is considered to have no identification significance when a reference genotype appears in the locus of the pig to be detected, and the detection locus is considered to have identification significance when a mutation genotype appears in the locus of the pig to be detected. For example, for a pig18-52722267 locus of pig A to be detected,

if the sequencing data is T, it is considered that pig A has no identification significance at pig 18-5272267 locus; if the sequencing data is C, it is considered that pig a has identification significance at pig18-52722267 locus.

Since there is false positive misjudgment when a single locus is used as the identification basis, and the misjudgment probability is high, the present invention uses the locus combination as the identification Marker.

Marker information of each breed identification locus combination is shown in Table 3.

TABLE 3
Information about Identification Marker Combination

| Breed | Marker | SNP combination |
|---|---|---|
| Pudong | Marker 1 | pig18-52722267, pig8-146130825, |
| white pig | | pig9-10041850 |
| | Marker 2 | pig18-52722267, pig8-146130825, |
| | | pig13-213464983 |
| | Marker 3 | pig18-52722267, pig9-10041850, |
| | | pig13-213464983 |
| | Marker 4 | pig8-146130825, pig9-10041850, |
| | | pig13-213464983 |

As shown in Table 3, Markers 1-4 are identification marker combinations for Pudong white pigs. For example, when the pig A to be detected has any one of the four marker combinations, it is considered that the pig A to be detected is a Pudong white pig.

The Pudong white pork products refer to Pudong white pig split meat and pickled products and cooked food products prepared from Pudong white pigs as raw materials.

Five ear tissue samples of pigs to be detected were randomly selected, and tissue DNA was extracted by a SDS method.

PCR amplification of DNA samples was carried out by primers.

The reaction system was template DNA of 1 ng/uL, a primer of 1 uL, H₂O of 3.8 uL and 2× Taq PCR masrer mix of 50 ul; and/or the reaction procedure of the PCR reaction was: pre-denaturation at 95C.° for 2 minutes, pre-denaturation at 95C.° for 30 seconds, annealing at 60° C. for 30 seconds, extension at 72C.° for 1 minute, with a cycle number of 30 times, and extension at 72C.° for another 10 min minutes.

The amplification results were detected by electrophoresis with 2% of an agarose gel and 1× TAE of a buffer solution as mediums. The gel electrophoresis conditions were: a current of 10 A, a voltage of 100v and time of 40 min. The amplification products of different primer pairs were compared with the standard product length in Table 1. If the product length is within the error range and consistent with the standard product length, the amplification result is considered qualified.

TABLE 4
SNP Polymorphism Analysis Table of Sample Sequencing Results

| Sample/SNP | REF | ALT | 1 | 2 | 3 | 4 | 5 |
|---|---|---|---|---|---|---|---|
| pig18-52722267 | T | C | √ | √ | | √ | |
| pig8-146130825 | G | T | | √ | √ | | √ |
| pig9-10041850 | G | A | | √ | √ | | √ |
| pig13-213464983 | C | T | | | | √ | √ |

In the table, REF represents a reference genotype, ALT represents a mutation genotype, and √ represents a detected mutation genotype.

As shown in Table 4, it can be seen from the sequencing data that if only a single locus is used as the identification basis, a pig to be detected belongs to multiple breeds at the same time.

Identification was carried out by using a Marker combination: mutations were detected at pig18-52722267 in a pig No. 1, which did not conform to Marker information, and the pig No. 1 was identified as not a Pudong white pig; mutations were detected at pig18-52722267, pig8-146130825, pig9-10041850 in a pig No. 2, which conformed to the Marker information, and the pig No. 2 was identified as a Pudong white pig; mutations were detected at pig8-146130825 and pig9-10041850 in a pig No. 3, which did not conform to the Marker information, and the pig No. 3 was identified as not a Pudong white pig; mutations were detected at pig18-52722267 and pig13-213464983 in a pig No. 4, which did not conform to the Marker information, and the pig No. 4 was identified as not a Pudong white pig; mutations were detected at pig8-146130825, pig9-10041850 and pig13-213464983 in pig No. 5, which conformed to Marker4, and the pig No. 5 was identified as a Pudong white pig.

At present, there are no patents related to the identification of Pudong white pigs and their meat products at home and abroad. The invention of the third-generation molecular markers to the identification of Pudong white pigs and their meat products has filled the blank in the market and effectively solved the problem of identification of Pudong white pigs. Compared with the existing patents that use the first-generation molecular markers (RFLP) and the second-generation molecular markers (SSR) to identify pig breeds, this identification method has the advantages of simpler operation, more accurate results, rapidness and high efficiency. Meanwhile, the third-generation molecular marker SNP is utilized in the present invention to overcome the defect of fewer available loci for the molecular markers of the previous two generations, and compared with the identification technology of the previous two generations of molecular markers, the identification method is also simplified.

The above specific implementation can be partially adjusted by those skilled in the art in different ways without departing from the principles and purposes of the present invention. The protection scope of the present invention is subject to the claims and not limited by the above specific implementation, and all embodiments within its scope are subject to the present invention.

What is claimed is:

1. A SNP marker combination for Pudong white pigs and raw meat products, comprising pig18-52722267, pig8-146130825, pig9-10041850 and pig13-213464983, wherein the pig18-52722267 represents a 52722266^{th} locus of pig chromosome No. 18, the pig8-146130825 represents a 146130825^{th} locus of pig chromosome No. 8, the pig9-10041850 represents a 10041850^{th} locus of pig chromosome No. 9, and the pig13-213464983 represents a 213464983^{th} locus of pig chromosome No. 13.

2. A method for identifying Pudong white pigs and raw meat products thereof based on the SNP marker combination according to claim 1, comprising: firstly extracting genomic DNAs of a raw pork or a meat product to be identified, then performing agarose gel electrophoresis and Sanger sequencing after PCR amplification to obtain information of each detection locus of the SNP marker combination, wherein a Pudong white pig and a meat product thereof are identified when specific mutations occur at any three loci of the SNP marker combination.

3. The method according to claim 2, wherein as for the PCR amplification, sequences of upstream and downstream primers of the pig18-52722267 are shown in SEQ ID No.1 and SEQ ID No.2, sequences of upstream and downstream primers of the pig8-146130825 are shown in SEQ ID No.3 and SEQ ID No.4, sequences of upstream and downstream primers of the pig9-10041850 are shown in SEQ ID No.5 and SEQ ID No.6, and sequences of upstream and downstream primers of the pig13-2134664983 are shown in SEQ ID No. 7 and SEQ ID No.8.

4. The method according to claim 2, wherein comparison information of identification loci of sequencing products is shown in the following table:

| SNP | REF | ALT |
|---|---|---|
| pig18-52722267 | T | C |
| pig8-146130825 | G | T |
| pig9-10041850 | G | A |
| pig13-213464983 | C | T |

wherein REF represents a reference genotype and ALT represents a mutation genotype.

5. The method according to claim 4, wherein the information of identification loci is: mutation genotypes and reference genotypes of detection loci; when a reference genotype appears in a detection locus of a pig to be detected, the locus is determined to have no identification significance; and when a mutation genotype appears in a detection locus of the pig to be detected, the locus is determined to have identification significance.


SEQUENCE LISTING (8 sequences)

SEQ ID NO: 1 - DNA, 21 bp, Artificial Sequence
  primer
gcgttttggggactcgtgata

SEQ ID NO: 2 - DNA, 21 bp, Artificial Sequence
  primer
actctgccgtttctcctccta

SEQ ID NO: 3 - DNA, 19 bp, Artificial Sequence
  primer
agaggagcggggtctttgc

SEQ ID NO: 4 - DNA, 24 bp, Artificial Sequence
  primer
cgttgtcaacttttgtctacctca

SEQ ID NO: 5 - DNA, 21 bp, Artificial Sequence
  primer
tagatgtgagccccagcagtt

SEQ ID NO: 6 - DNA, 19 bp, Artificial Sequence
  primer
acttctgctccccgcaccg

SEQ ID NO: 7 - DNA, 23 bp, Artificial Sequence
  primer
cactcaggatattcacaatctgg

SEQ ID NO: 8 - DNA, 21 bp, Artificial Sequence
  primer
ttaaaacgaccacggcaactc